(19) United States
(12) Patent Application Publication
(10) Pub. No.: US 2026/0330731 A1
(43) Pub. Date: Oct. 22, 2026

(54) Method for phase-contrast-based magnetic resonance image reconstruction

(71) Applicant: University of Iowa Research Foundation, Iowa City, IA (US)

(72) Inventor: Xi Peng, Iowa City, IA (US)

(73) Assignee: University of Iowa Research Foundation, Iowa City, IA (US)

(21) Appl. No.: 19/652,634

(22) Filed: Apr. 20, 2026

Related U.S. Application Data
(60) Provisional application No. 63/792,053, filed on Apr. 21, 2025.

(51) Int. Cl. G06T 12/20; G06T 7/00; G16H 50/20

(52) U.S. Cl. CPC G06T 12/20; G06T 7/0012; G16H 50/20; G06T 2207/10088; G06T 2207/20081; G06T 2207/20084; G06T 2207/30016; G06T 2207/30096; G06T 2210/41; G06T 2211/441; G06T 2211/461

(57) ABSTRACT

A method for phase-contrast magnetic resonance image reconstruction that improves image quality while reducing acquisition time. The method receives undersampled data from multiple phase-contrast magnetic resonance image acquisitions and initializes a deep generative neural network with network parameters and acquisition-specific input vectors. Through an iterative process, the network parameters and input vectors are modified to minimize a cost function including a data consistency term, an anatomical consistency term enforcing structural similarity across acquisitions, and a physics-based term enforcing properties of phase variations. The modified network then generates high-quality reconstructed phase-contrast magnetic resonance images without requiring pre-acquired training datasets, enabling faster clinical implementation of phase-contrast MRI applications including elastography, flow imaging, and thermometry.

### RELATED APPLICATIONS

This application claims priority to U.S. Provisional Patent Application No. 63/792,053 filed Apr. 21, 2025, hereby incorporated by reference in its entirety.

### FIELD OF THE DISCLOSURE

The present disclosure relates to phase-contrast magnetic resonance imaging (MRI). More particularly, but not exclusively, the present disclosure relates to a method for phase-contrast-based magnetic resonance image reconstruction.

### BACKGROUND

Phase-contrast magnetic resonance imaging (MRI) is a special type of MRI technique that encodes physiological information into the phase of MR images, such as MR elastography, MR thermometry and MR flow imaging, encoding mechanical vibration, temperature and blood flow respectively.

Data acquisition is usually long because it requires repeated sampling of the same image volume at different times and along different directions to fully capture the variation of vibration, temperature and blood flow. Therefore, it is often difficult to achieve high resolution, high signal-to-noise-ratio and fast scan practically, limiting their clinical application.

What is needed is a new approach to achieve high quality phase-contrast MR image reconstruction from highly under sampled data, reducing scan time and allowing practical implementation.

### SUMMARY

Therefore, it is a primary object, feature, or advantage to improve over the state of the art.

It is a further object, feature, or advantage of the present disclosure to improve phase-contrast magnetic resonance imaging (MRI) by leveraging the “static” anatomical information underlying the repeated acquisitions to reduce acquisition.

It is a still further object, feature, or advantage of the present disclosure to incorporate such prior information into the image reconstruction by using a deep generative neural network to efficiently represent the spatial-temporal image function, along with priors to enforce the “shared” anatomical information.

Another object, feature, or advantage is to improve phase-contrast MRI by a new deep-learning reconstruction method that does not require any training data set.

Yet another object, feature, or advantage is to incorporate phase-contrast-specific prior information to further improve the reconstruction quality.

It is a further object, feature, or advantage of the present disclosure to reduce acquisition time for phase-contrast MRI while maintaining or improving image quality.

It is a still further object, feature, or advantage of the present disclosure to implement the reconstruction method without requiring large pre-acquired training datasets, unlike conventional deep learning approaches.

It is another object, feature, or advantage to enable higher resolution phase-contrast MRI in clinical settings where time constraints previously limited its application.

It is yet another object, feature, or advantage to leverage the inherent redundancy in anatomical structures across multiple phase-contrast acquisitions.

It is a further object, feature, or advantage to adaptively learn image representation directly from undersampled data without supervision.

It is a still further object, feature, or advantage to produce quantitative biomarkers with improved accuracy and precision from reconstructed images.

It is another object, feature, or advantage to enable more reliable clinical diagnosis through higher quality phase maps and derived measurements.

It is yet another object, feature, or advantage to incorporate domain-specific physics knowledge into the reconstruction process.

It is a further object, feature, or advantage to provide flexibility across different phase-contrast applications including elastography, flow imaging, and thermometry.

It is a still further object, feature, or advantage to improve patient comfort by reducing scan time while maintaining diagnostic quality.

It is another object, feature, or advantage to maintain consistent anatomical information while accurately representing dynamic phase changes.

It is yet another object, feature, or advantage to enhance diagnostic capability for various neurological conditions including brain tumors, Alzheimer's disease, multiple sclerosis, and Parkinson's disease.

One or more of these and/or other objects, features, or advantages of the present disclosure will become apparent from the specification and claims that follow. No single embodiment need provide each and every object, feature, or advantage. Different embodiments may have different objects, features, or advantages. Therefore, the present disclosure is not to be limited to or by any objects, features, or advantages stated herein.

According to one aspect, a method for phase-contrast magnetic resonance image reconstruction that receives undersampled data from multiple phase-contrast magnetic resonance image acquisitions is provided. The method initializes a deep generative neural network with network parameters and acquisition-specific input vectors. It then iteratively modifies these network parameters and input vectors to minimize a cost function. In a special case, the input vector can be fixed as the spatiotemporal coordinates of the targeted image function. This cost function includes a data consistency term between the undersampled data and the network output, an anatomical consistency term that enforces structural similarity across the multiple acquisitions, and a physics-based term that enforces properties of phase variations. The method finally generates reconstructed phase-contrast magnetic resonance images using the modified network.

The anatomical consistency term in the method may penalize differences between magnitude images from different acquisitions. The physics-based term may enforce at least one of spatial and/or temporal smoothness of phase components, divergence-free properties of vector fields, or harmonic properties of displacement fields. The cost function may further include an input vector constraint term. Each of the multiple phase-contrast magnetic resonance image acquisitions may represent a different time point, a different encoding direction, or a different phase of motion.

The deep generative neural network in the method may map between a low-dimensional latent space and image space. The method may be applied to magnetic resonance elastography, magnetic resonance flow imaging, or magnetic resonance thermometry. The method may also include extracting quantitative physiological information from the reconstructed phase-contrast magnetic resonance images and providing these quantitative physiological measurements for clinical diagnosis. The physics-based term may include application-specific properties, such as properties of mechanical wave propagation for elastography, properties of fluid dynamics for flow imaging, or properties of heat diffusion for thermometry. The iterative modification process may alternate between updating the network parameters while holding the input vectors fixed and updating the input vectors while holding the network parameters fixed. The method may be implemented using a non-transitory computer-readable medium storing instructions that cause a computing device to perform steps of the method. The deep generative neural network may represent an image at a specific acquisition as a function of the parameters and an acquisition-specific input vector. The iterative modification of the network parameters and input vectors may involve an unsupervised learning process that estimates the parameters and input vectors directly from the undersampled data.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Illustrated embodiments of the disclosure are described in detail below with reference to the attached drawing figures, which are incorporated by reference herein.

FIG. 1 is an illustration of the proposed deep generative model for improved MRE image reconstruction.

FIG. 2 and FIG. 3 are comparisons between fully sampled reconstruction and under-sampled reconstruction from SENSE, Low-rank, and the proposed methods. The proposed method was able to produce high-quality MRE images better delineating anatomical details along with comparable displacement and stiffness estimation as the fully sampled reconstruction.

FIG. 4 is a quantitative comparison of median stiffness in white and gray matters. The proposed method produced stiffness measurements comparable to the fully sampled data and across all undersampling cases (e.g., varying number of spiral arms).

FIG. 5 is another comparison between reconstructions from SENSE, deep generative (DeepGM) and the proposed method.

FIG. 6 illustrates magnitude images and stiffness quantifications from different methods with the proposed method producing images with sharper structural appearance and better quality stiffness maps.

FIG. 7 is a flow chart illustrating a method for phase-contrast magnetic resonance image reconstruction according to an embodiment of the present disclosure.

FIG. 8 is a block diagram illustrating a system for phase-contrast magnetic resonance image reconstruction according to an embodiment of the present disclosure.

### DETAILED DESCRIPTION

The following detailed description is merely exemplary in nature and is not intended to limit the present disclosure or the application and uses of the present disclosure. Furthermore, there is no intention to be bound by any theory presented in the preceding background or the following detailed description.

The present disclosure addresses the need for a deep learning method for improved phase-contrast MR image reconstruction without the requirement of high-quality training datasets. The features include the use of deep generative neural network to represent the spatial-temporal image function and incorporation of regularization terms that impose the “shared” static anatomical information and enforce the known physics of the dynamically varying component in the image phase.

The present disclosure includes a new deep-learning-based approach to achieve high quality phase-contrast MR image reconstruction from highly undersampled data, thus reducing scan time and allowing practical implementation. The “static” anatomical information underlying the repeated acquisitions are highly redundant and can be leveraged to reduce acquisition time; while the “dynamic” component in the varying image phase obeys certain prior known physics property of the underlying directional vibration, flow or temperature dynamics. The method provides for incorporating such prior information into the image reconstruction by using a deep generative neural network to efficiently represent the spatial-temporal image function, along with priors to enforce the “shared” anatomical information and the known vibration, flow or temperature properties.

The reconstruction method may be implemented on any computational server that receives raw MR data from the MR scanners and return high quality MR images for phase-contrast based MR applications including but not limited to MR elastography, MR thermometry and MR 4D flow.

Conventional under sampling methods, such as parallel imaging, compressed sensing, and low-rank modeling [1], have been exploited to enable fast acquisition by leveraging spatial and temporal redundancies. Recently, deep learning (DL)-based reconstructions have shown considerable promise in producing high quality MR images from under sampled data. However, DL-based reconstruction for phase-contrast MR application is underdeveloped probably due to 3D high-quality phase-contrast MR datasets being difficult to acquire for training supervised models.

The present disclosure includes a deep generative model with an unsupervised learning scheme, which had been developed in dynamic imaging [2-5] and parameter mapping [6], for improved phase-contrast MR image reconstruction. Novel physics-specific (such as mechanical vibration, flow or temperature dynamics) priors were incorporated to improve and stabilize the training of the generative network and enhance SNR.

The nonlinear representation of the phase-contrast MR image series can be expressed as:

βₜ=U(νₜ,θ),

where ρₜ is the MR image at the t-th repetition (i.e., different time points or directions), U denotes the generative network with learnable weights θ and input vector νₜ for the t-th repetition. The main assumption is that different phase-contrast repetitions share similar anatomical information that can be well represented by the same generative model, while phase-dependent variations (e.g., vibration, flow, temperature, etc.) reside in a low-dimensional space that can be well captured and decoded from the input vectors. To this end, the reconstruction of phase-contrast MR images is equivalent to estimate θ and νₜ.

We propose to estimate them by minimizing the following cost function:

$${arg min}_{θ , {v}_{t}} {\sum }_{t = 1}^{T} {‖ {d}_{t} - E {ρ}_{c} ‖}_{2}^{2} + {λ}_{1} {R}_{anat} ( ρ ) + {λ}_{2} {R}_{phase} ( ρ ) + {λ}_{3} {R}_{v} ( ν ) ,$$

where E represents the forward encoding operator of the MRI system including the Fourier operation, coil sensitivity function and field homogeneity, etc. dₜ is the raw MR data in k-space. Rₐₙₐₜ, Rₚₕₐₛₑ and R_{ν} are the regularization terms enforcing prior information on the shared anatomical structure, vibration/flow/temperature-induced phase variations, and input vectors, respectively. ρ={ρ₁, ρ₂, . . . , ρ_{T}} and ν={ν₁, ν₂, . . . , ν_{T}} are sets of ρₜ and νₜ. For instance, in one implementation, the regularizations can be selected as:

$${R}_{anat} ( ρ ) = {\sum }_{i , j , i \neq j} {‖ \vert {ρ}_{i} \vert - \vert {ρ}_{j} \vert ‖}_{1}$$

where Rₐₙₐₜ enforces the anatomical similarity between the magnitude information of any image repetitions. R_{ν} can be selected as simple as:

$${R}_{v} ( v ) = {‖ v ‖}_{2}^{2}$$

where R_{ν} impose the L2 regularization on the input vector, or:

$${R}_{v} ( {v}_{i} , {v}_{j} ) = {\sum }_{i , j , i \neq j} {‖ {v}_{i} - {v}_{j} ‖}_{1}$$

where R_{ν} penalizes the distance between input vectors corresponding to different vibration/flow/temperature state. In a special case, the input vector can be fixed as the spatiotemporal coordinates of the targeted image function. For MRE and flow imaging, Rₚₕₐₛₑ(ρ) can be chosen as:

$${R}_{phase} ( ρ ) = TV ( H ρ ) + α {‖ Div ( ρ ) ‖}_{2}^{2}$$

where Rₚₕₐₛₑ enforces smoothness and “free”-divergence of the 3D temporal varying vector field, e.g., vibration displacement or blood flow. TV represents the total variation, Div calculates the divergence of the 3D vector field from the MRE/flow images. For MRE, operator H first computes the wave-dependent phase components and then extracts the harmonic displacement at the frequency of interest; For flow imaging, H computes the flow-dependent phase components. Lastly, for MR thermometry, Rₚₕₐₛₑ (ρ) can be chosen as:

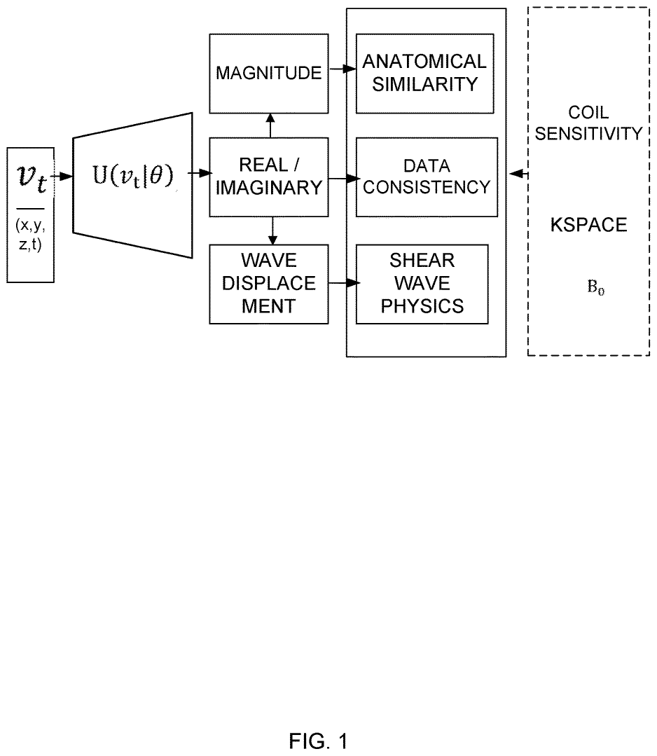

Rₚₕₐₛₑ(ρ)=TV(Hρ)

where H computes the temperature-dependent phase components from the phase-contrast MR images. A pictorial illustration of the method is shown in FIG. 1.

We evaluated the proposed method using a fully sampled multi-slice spin-echo spiral MRE dataset (TR/TE=5000/50 ms, flip angle=90°, scan time=10 minutes, 13.5 ms spiral readout, 5 spiral arms, 2 mm isotropic resolution) and an in-plane fully sampled 3D gradient-echo spiral MRE dataset (TR/TE=66.67/35 ms, FA=23°, 1.2× slice oversampling, 2× through-plane uniform undersampling, scan time=5 minutes, 13.6 ms spiral readout, 5 spiral arms, 2 mm isotropic resolution), both of which were acquired on a 3 T MR scanner with a 14-channel head coil array and same MRE settings (4 MRE phase-offsets, 6 directions, 60 Hz shear wave frequency).

The network was constructed in Pytorch and trained using stochastic gradient descent with an Adam optimizer on one Nvidia A100 80 GB GPU for a maximum of 500 iterations. Initial input vectors were computed using SVD from only the central portion of k-space. The coil-sensitivity and field inhomogeneity maps were obtained separately using low-resolution pre-scans. Given the reconstructed images, the curl operation was conducted to extract the shear wave component of the 3D vector field followed by a standard direction inversion of the Helmholtz equation to produce the stiffness maps.

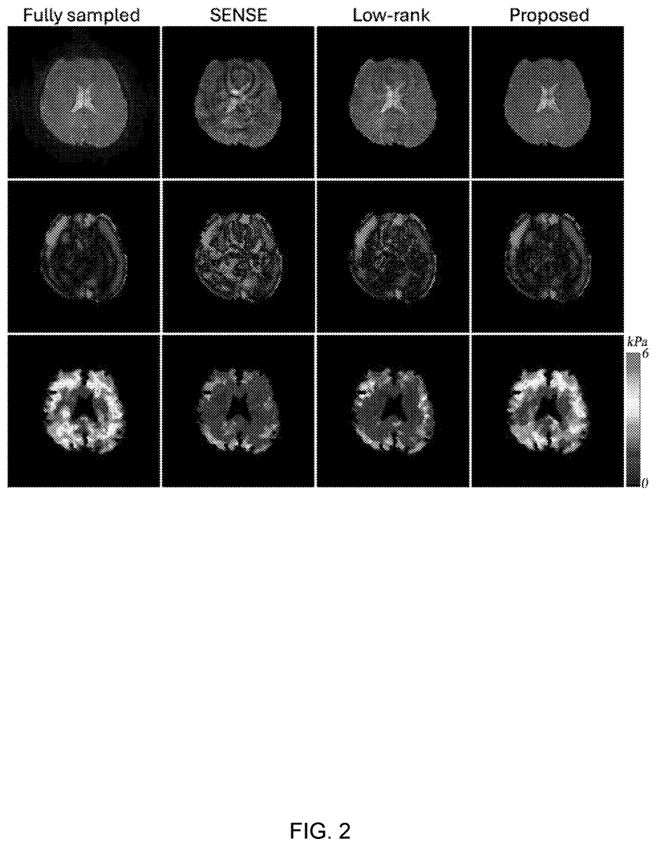

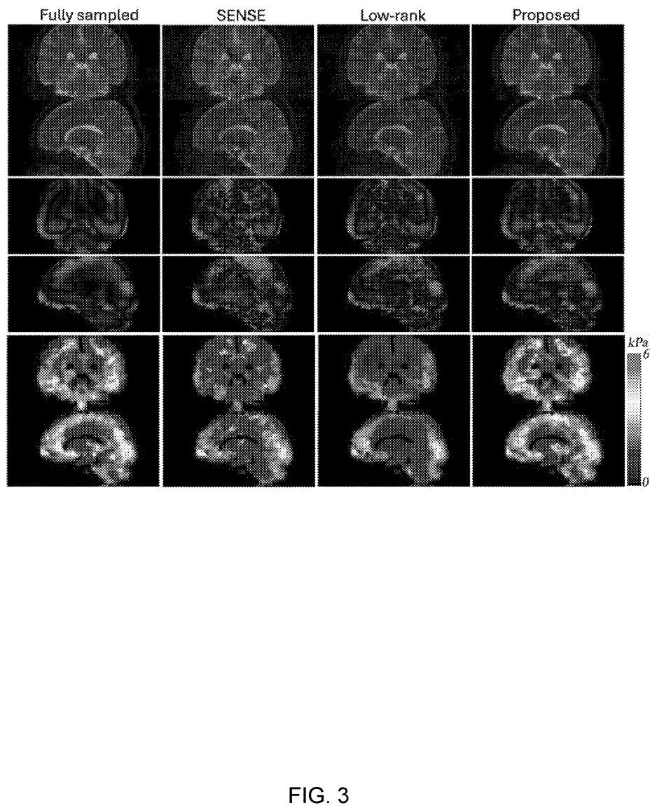

Results are shown in FIG. 2 through FIG. 6. FIG. 2 and FIG. 3 are comparisons between fully sampled reconstruction and under-sampled (i.e., 5× in-plane acceleration) reconstruction from SENSE, Low-rank, and the proposed methods using the gradient-echo and spin-echo datasets respectively. The proposed method was able to produce high-quality MRE images better delineating anatomical details along with comparable displacement and stiffness estimation as the fully sampled reconstruction.

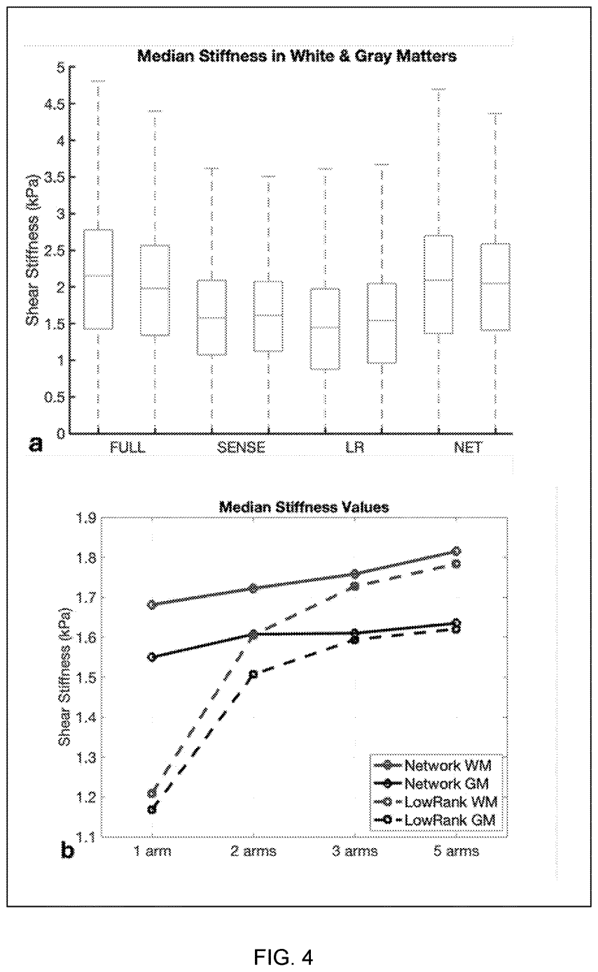

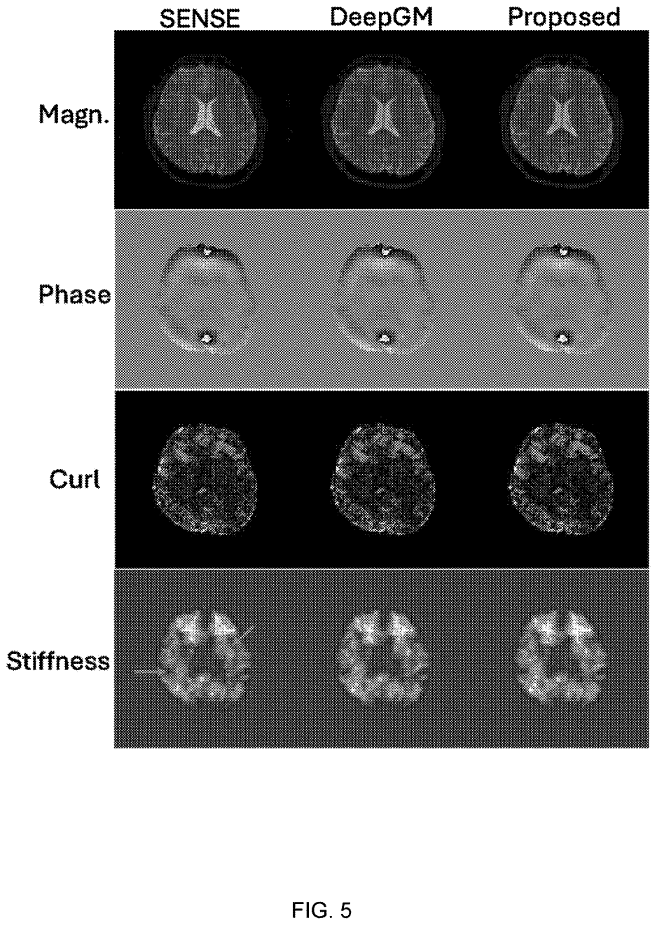

FIG. 4 is a quantitative comparison of median stiffness in white and gray matters. The proposed method produced stiffness measurements comparable to the fully sampled data and across all undersampling cases (e.g., varying number of spiral arms). FIG. 5 is another comparison between reconstructions (i.e., 1.67× in-plane acceleration) from SENSE, deep generative model (DeepGM) and the proposed method using the spin-echo dataset.

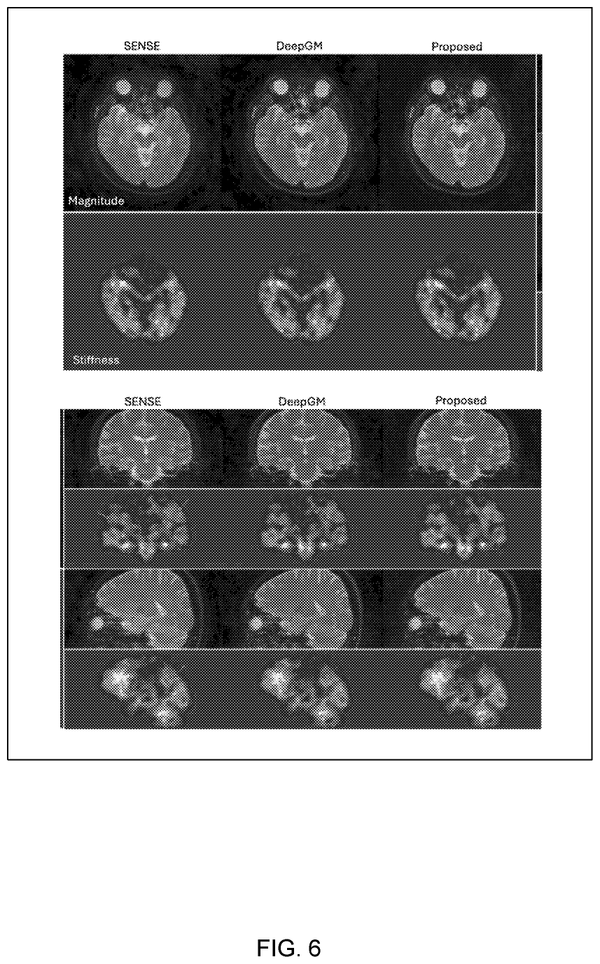

FIG. 6 illustrates reconstructed magnitude images and stiffness quantifications at an in-plane acceleration factor of 1.67 from different methods with the proposed method producing images with sharper structural appearance and better quality stiffness maps.

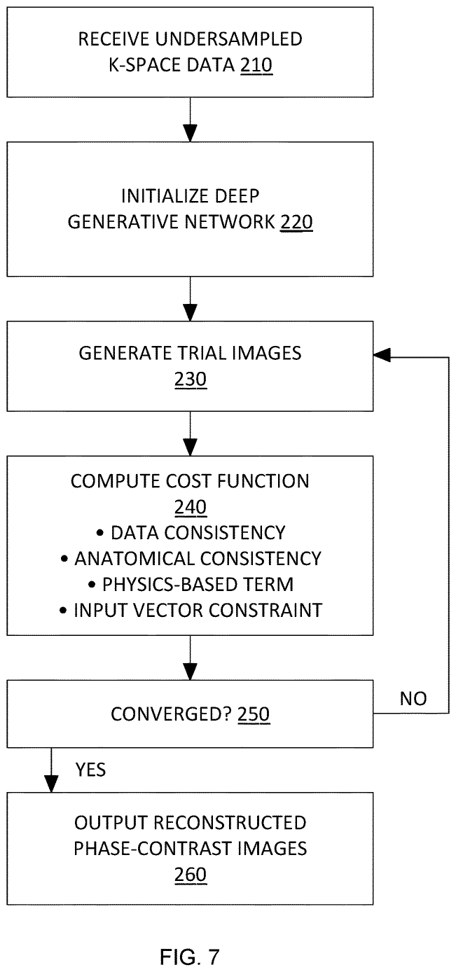

Referring now to FIG. 7, a flow chart illustrating a method for phase-contrast magnetic resonance image reconstruction according to the preferred embodiments is shown. The method may be performed by a computing device comprising one or more processors and may operate on data acquired by a magnetic resonance imaging scanner.

At step (210), the computing device receives undersampled k-space data from a plurality of phase-contrast magnetic resonance image acquisitions. The undersampled data may be received directly from a magnetic resonance imaging scanner interface, loaded from previously acquired raw data files, or accessed from raw data transmitted over a network or stored in a cloud-based repository. Each of the plurality of phase-contrast magnetic resonance image acquisitions may represent a different time point, a different encoding direction, or a different phase of motion. The data may be acquired using various sampling trajectories including spiral sampling, Cartesian undersampling, radial undersampling, or other non-Nyquist sampling strategies. In some embodiments, the undersampled data corresponds to an acceleration factor of at least two relative to Nyquist sampling requirements for the desired image resolution.

At step (220), the computing device initializes a deep generative neural network with network parameters θ and a set of acquisition-specific input vectors νₜ. The deep generative neural network maps between a low-dimensional latent space defined by the input vectors and a higher-dimensional image space. In some embodiments, the deep generative neural network may comprise a convolutional neural network, a fully-connected network, a variational autoencoder, a normalizing flow, a diffusion model, an implicit neural representation, or other parametric function approximator. The network parameters θ may be initialized randomly or according to a predetermined initialization scheme. The acquisition-specific input vectors νₜ may be initialized using singular value decomposition applied to a central portion of the k-space data, or may be initialized randomly. Each input vector νₜ corresponds to one of the plurality of phase-contrast magnetic resonance image acquisitions, such that the deep generative neural network represents an image at a specific acquisition as a function of the network parameters θ and the corresponding acquisition-specific input vector νₜ. In a special case, the input vector can be fixed as the spatiotemporal coordinates of the targeted image function.

At step (230), the computing device generates trial images by processing the set of acquisition-specific input vectors through the deep generative neural network. For each acquisition index t, the network produces a trial image ρₜ=U(νₜ|θ), where U denotes the deep generative neural network. The network parameters θ are shared across all acquisitions, while each acquisition uses its own input vector νₜ. The shared network parameters encode anatomical information that is common across all acquisitions, while the acquisition-specific input vectors capture phase-dependent variations such as vibration displacement, blood flow velocity, or temperature change.

At step (240), the computing device computes a cost function value based on the trial images and the received undersampled data. The cost function comprises a plurality of terms. A data consistency term measures the fidelity between the undersampled k-space data and the trial images projected into k-space through a forward encoding operator of the magnetic resonance imaging system. The forward encoding operator may include a Fourier operation, coil sensitivity functions, and field inhomogeneity corrections. An anatomical consistency term enforces structural similarity across the plurality of acquisitions by penalizing differences between magnitude images from different acquisitions. In some embodiments, the anatomical consistency term may compute an L1 norm of the difference between the magnitude of any two trial images from different acquisitions. A physics-based term enforces properties of phase variations derived from known physical laws governing the phenomenon of interest. The physics-based term may enforce at least one of: spatial smoothness or temporal smoothness of phase components; divergence-free properties of vector fields; or harmonic properties of displacement fields. For magnetic resonance elastography, the physics-based term may enforce properties of mechanical wave propagation, including total variation smoothness of harmonic displacement components and a divergence-free constraint on the three-dimensional displacement vector field. For magnetic resonance flow imaging, the physics-based term may enforce properties of fluid dynamics, including smoothness and divergence-free properties of the blood flow velocity field. For magnetic resonance thermometry, the physics-based term may enforce properties of heat diffusion, including total variation smoothness of temperature-dependent phase components. The cost function may further comprise an input vector constraint term that regularizes the input vectors, for example by imposing an L2 norm penalty on the input vectors or by penalizing distances between input vectors corresponding to different acquisition states.

At step (250), the computing device determines whether the iterative optimization has converged. Convergence may be determined based on a predetermined maximum number of iterations, a threshold on the change in cost function value between successive iterations, a threshold on the gradient magnitude, or other stopping criteria. If convergence has not been reached, the method returns to step (230) and repeats the generation of trial images with updated network parameters and input vectors. The update may be performed using stochastic gradient descent, an Adam optimizer, LBFGS, conjugate gradient methods, or other optimization algorithms. In some embodiments, the iterative modification may alternate between updating the network parameters θ while holding the input vectors νₜ fixed, and updating the input vectors νₜ while holding the network parameters θ fixed. The iterative modification constitutes an unsupervised learning process that estimates the network parameters and input vectors directly from the undersampled data without requiring any pre-acquired training datasets. In a special case, the input vector can be fixed as the spatiotemporal coordinates of the targeted image function.

At step (260), upon convergence, the computing device outputs reconstructed phase-contrast magnetic resonance images generated by the modified deep generative neural network. The reconstructed images may be used for clinical diagnosis or further quantitative analysis. In some embodiments, the method further comprises extracting quantitative physiological information from the reconstructed phase-contrast magnetic resonance images, such as tissue stiffness maps for elastography, flow velocity maps for flow imaging, or temperature maps for thermometry. The quantitative physiological measurements may then be provided for clinical diagnosis of conditions including but not limited to brain tumors, Alzheimer's disease, multiple sclerosis, Parkinson's disease, and normal pressure hydrocephalus.

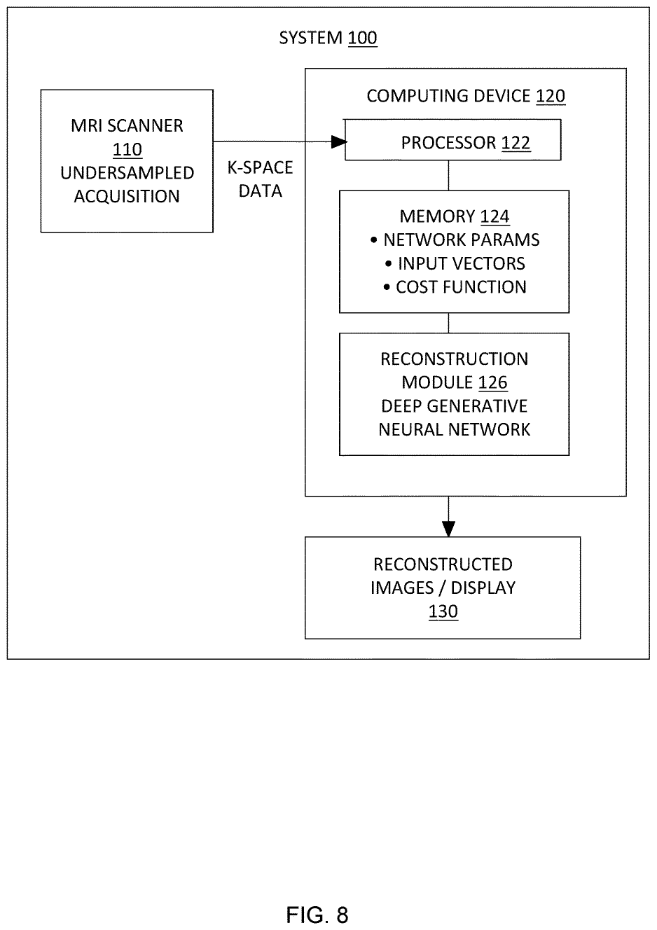

Referring now to FIG. 8, a block diagram illustrating a system (100) for phase-contrast magnetic resonance image reconstruction according to the preferred embodiments is shown.

The system (100) comprises a magnetic resonance imaging scanner (110) and a computing device (120). The magnetic resonance imaging scanner (110) is configured to acquire undersampled k-space data from a subject. The magnetic resonance imaging scanner (110) may be any clinical or research MRI system operating at various field strengths including but not limited to 1.5 T, 3 T, or 7 T. The magnetic resonance imaging scanner (110) may employ various pulse sequences for phase-contrast acquisition, including spin-echo spiral sequences, gradient-echo sequences, echo-planar imaging sequences, or other suitable acquisition sequences. The magnetic resonance imaging scanner (110) may include multi-channel receiver coil arrays, such as a head coil array, body coil array, or other application-specific coil arrays.

The computing device (120) is coupled to the magnetic resonance imaging scanner (110) and is configured to receive the undersampled k-space data and perform image reconstruction. The computing device (120) need not be physically co-located with the magnetic resonance imaging scanner (110) and may be connected to the scanner via a local network, a wide area network, or a cloud-based infrastructure. In some embodiments, the computing device (120) may be an on-scanner reconstruction computer integrated directly with the magnetic resonance imaging scanner (110). In other embodiments, the computing device (120) may be a remote server, a GPU compute cluster, a cloud computing instance, or other computational platform.

The computing device (120) comprises a processor (122), a memory (124), and a reconstruction module (126). The processor (122) may include one or more central processing units, graphics processing units, tensor processing units, or other processing hardware configured to execute the computational operations described herein. In some embodiments, the processor (122) comprises a graphics processing unit configured for parallel numerical computation, such as an Nvidia A100 or other general-purpose GPU.

The memory (124) is coupled to the processor (122) and stores data and instructions for the reconstruction process. The memory (124) may store the network parameters θ of the deep generative neural network, the acquisition-specific input vectors Ut (or spatiotemporal coordinates), cost function definitions and hyperparameters, coil sensitivity maps, field inhomogeneity maps, and the received undersampled k-space data. The memory (124) may comprise random access memory, high-bandwidth memory associated with a GPU, solid-state storage, or combinations thereof.

The reconstruction module (126) implements the deep generative neural network and the iterative optimization process. The reconstruction module (126) may be implemented as software instructions stored in the memory (124) and executed by the processor (122), as firmware, as a hardware accelerator, or as combinations thereof. In some embodiments, the reconstruction module (126) may be implemented using a deep learning framework such as PyTorch, TensorFlow, JAX, or other suitable frameworks. The reconstruction module (126) is configured to initialize the deep generative neural network with network parameters and acquisition-specific input vectors, iteratively modify the network parameters and input vectors to minimize a cost function comprising a data consistency term, an anatomical consistency term, and a physics-based term as described with reference to FIG. 7, and generate reconstructed phase-contrast magnetic resonance images using the modified network.

The reconstructed images and any derived quantitative measurements are output to a display or storage device (130). The display or storage device (130) may include a clinical workstation display, a picture archiving and communication system, a network-attached storage device, or other output device. In some embodiments, the reconstructed images are transmitted to a clinical information system for integration with a patient record. In some embodiments, quantitative physiological measurements derived from the reconstructed images, such as tissue stiffness maps, flow velocity maps, or temperature maps, are also output to the display or storage device (130) for use in clinical diagnosis.

Therefore, methods have been shown and described for a new deep-learning reconstruction method for phase-contrast MRI that does not require any training dataset. The method and system allow for incorporation of phase-contrast-specific prior information to further improve the reconstruction quality. These priors include the “shared” anatomical information and the known physics of temporal dynamics, such as mechanical vibration, blood flow and temperature.

From a technical standpoint, the preferred embodiments may provide several computational and imaging advantages over conventional approaches. Conventional supervised deep learning reconstruction methods may require pre-acquired training datasets comprising hundreds or thousands of fully-sampled three-dimensional volumes, which may consume substantial storage resources and computational time for training. The preferred embodiments may eliminate this requirement by performing unsupervised, instance-adaptive reconstruction, potentially reducing the computational pipeline from a multi-stage training-then-inference workflow to a single optimization performed directly on the acquired data. Additionally, the preferred embodiments may reduce MRI scanner acquisition time by enabling reconstruction from undersampled data, which may improve patient throughput and comfort. The incorporation of physics-based regularization may improve signal-to-noise ratio in the reconstructed images without requiring additional scan time, potentially enhancing the quality of derived quantitative biomarkers such as tissue stiffness maps. A technically significant aspect of the preferred embodiments is the ability to perform high-quality image reconstruction.

A technically significant aspect of the preferred embodiments is the ability to perform high-quality image reconstruction without requiring any pre-acquired training datasets. Conventional supervised deep learning reconstruction methods may require training datasets that are impractical to acquire for three-dimensional phase-contrast MRI protocols due to the long scan times involved. The preferred embodiments may overcome this limitation by formulating reconstruction as an unsupervised optimization problem in which both the network parameters and the acquisition-specific input vectors are estimated directly from the undersampled measurements acquired for the subject at hand. This may be described as an “instance-adaptive” or “scan-specific” reconstruction, in which the network learns a representation tailored to the specific anatomy and physiological state of the subject being scanned, rather than relying on population-level statistics derived from a training cohort. This instance-adaptive property may be particularly advantageous for subjects with anatomical abnormalities or physiological states that are underrepresented in available training datasets, potentially improving reconstruction quality and diagnostic utility in precisely the cases where accurate imaging is most clinically important.

The methods and systems may be used in any number of different applications including any clinical and basic science applications where phase-contrast MRI scans, such as MR elastography, flow imaging, MR thermometry are used and can provide the biomarkers of interest, including but not limited to brain tumor, Alzheimer's disease, multiple sclerosis, Parkinson's disease, normal pressure hydrocephalus, etc.

Various alternatives and variations may be implemented without departing from the spirit of the disclosure. For instance, the deep generative neural network may utilize different architectural designs including various depths, widths, or types of layers such as convolutional, fully-connected, or transformer-based configurations. The cost function may incorporate additional or alternative regularization terms tailored to specific clinical applications. The optimization strategy may employ various algorithms beyond stochastic gradient descent, such as LBFGS, conjugate gradient methods, or adaptive learning rate schemes. Moreover, the initialization strategy for network parameters and input vectors may be modified based on prior knowledge about the anatomical structures or phase variations expected in particular applications. In a special case, the input vector can be fixed as the spatiotemporal coordinates of the targeted image function.

The method may be implemented on various computational platforms ranging from dedicated hardware accelerators, cloud-based infrastructure, to on-scanner reconstruction modules integrated directly with MRI systems. The preferred embodiments may include additional high-dimensional MRI applications beyond those explicitly described, including functional MRI, diffusion imaging, or emerging techniques that encode physiological information in the phase of MR images.

As used herein, the term “coupled” or “connected” may refer to direct connection or indirect connection through one or more intermediary components.

As used herein, the term “module” or “component” may refer to hardware, software, firmware, or combinations thereof configured to perform the described functions.

As used herein, the singular forms “a,” “an,” and “the” include plural referents unless the context clearly dictates otherwise.

As used herein, the term “or” is inclusive, meaning “and/or” unless the context clearly indicates otherwise.

As used herein, the term “based on” means “based at least in part on” unless expressly specified otherwise.

As used herein, the terms “comprises,” “comprising,” “includes,” “including,” “has,” “having,” or any other variation thereof are intended to cover non-exclusive inclusions.

As used herein, the term “processor” refers to any hardware, firmware, or combination thereof capable of executing computational instructions or performing numerical operations, and includes without limitation: central processing units (CPUs); graphics processing units (GPUs), including general-purpose GPUs (GPGPUs) configured for parallel numerical computation; tensor processing units (TPUs) or other application-specific integrated circuits (ASICs) designed to accelerate matrix and tensor operations used in neural network training and inference; neural processing units (NPUs) or dedicated AI accelerators, including but not limited to those designed for deep learning workloads; field-programmable gate arrays (FPGAs) configured to implement neural network computations; neuromorphic computing devices; distributed processing systems comprising multiple interconnected processors operating in parallel; cloud-based processing infrastructure accessed via a network interface; and any combination of the foregoing. The term “processor” is not limited to any single physical device and expressly encompasses multi-processor architectures, processor clusters, and heterogeneous computing environments in which different types of processors handle different aspects of a computation. For the avoidance of doubt, a system in which a GPU or AI accelerator performs neural network forward and backward passes while a CPU manages data loading and optimization bookkeeping constitutes a single “processor” as used herein.

As used herein, the term “non-transitory computer-readable medium” includes all computer-readable media except for transitory, propagating signals.

As used herein, the term “computing device” refers to any device or system comprising one or more processors as defined herein, along with associated memory and input/output interfaces, configured to execute the computational operations described herein. The term includes without limitation: standalone workstations; server systems; GPU compute clusters; cloud computing instances; on-scanner reconstruction computers integrated with a magnetic resonance imaging system; edge computing devices; and combinations of the foregoing operating as a unified system. A “computing device” need not be physically co-located with the magnetic resonance imaging scanner from which it receives data and may be connected to such scanner via a network.

As used herein, the term “deep generative neural network” refers to any neural network architecture capable of generating data by mapping from a low-dimensional latent or input space to a higher-dimensional data space, including but not limited to: convolutional networks, fully-connected networks, variational autoencoders, generative adversarial networks, normalizing flows, diffusion models, implicit neural representations, or other parametric function approximators. The term is not limited to any particular architectural choice.

As used herein, the term “undersampled data” refers to k-space data acquired with fewer measurements than required by the Nyquist sampling theorem for the desired image resolution, including but not limited to data acquired via Cartesian undersampling, spiral undersampling, radial undersampling, or other non-Nyquist sampling strategies.

As used herein, the term “phase-contrast magnetic resonance imaging” refers to any MRI technique that encodes physiological information into the phase of the complex-valued MR image, including but not limited to MR elastography, MR flow imaging (including 4D flow MRI), MR thermometry, and other phase-sensitive MRI techniques.

As used herein, the term “physics-based term” refers to any regularization penalty derived from known physical laws or principles governing the phenomenon of interest, including but not limited to properties of mechanical wave propagation, fluid dynamics, heat diffusion, electromagnetism, or other physical processes. The term is not limited to a particular mathematical formulation.

As used herein, the term “receiving undersampled k-space data” refers to obtaining, from a magnetic resonance imaging scanner or from a data store populated by a magnetic resonance imaging scanner, k-space measurements acquired with fewer samples than required by the Nyquist criterion for the desired image resolution. The term encompasses direct receipt of data from a scanner interface, loading of previously acquired raw data files, and access to raw data transmitted over a network or stored in a cloud-based repository. In each case, the data is understood to have been physically generated by a magnetic resonance imaging scanner through interaction of radiofrequency pulses with nuclear spins in a subject, and the reconstruction methods described herein are specifically designed for and operate on data having the structure, noise properties, and physical encoding characteristics of MRI k-space measurements.

The present disclosure is not to be limited to the particular embodiments described herein. In particular, the present disclosure contemplates numerous variations in the network architecture, cost function formulations, regularization terms, optimization algorithms, implementation platforms, and clinical applications. The foregoing description has been presented for purposes of illustration and description. It is not intended to be an exhaustive list or limit any of the present disclosure to the precise forms disclosed. It is contemplated that other alternatives or exemplary aspects are considered included. The description is merely examples of embodiments, processes or methods. It is understood that any other modifications, substitutions, and/or additions can be made, which are within the intended spirit and scope of the present disclosure.

### REFERENCES

[1] McIlvain G, Cerjanic A M, Christodoulou A G, McGarry M D J, Johnson C L. OSCILLATE: A low-rank approach for accelerated magnetic resonance elastography. Magn Reson Med. 2022; 88 (4): 1659-1672.
[2] Yaman B, et al. Self-supervised learning of physics-guided reconstruction neural networks without fully sampled reference data, Magn. Reson. Med. 2020; 84:3172-3191.
[3] J Yoo, et al, Time-Dependent Deep Image Prior for Dynamic MRI. IEEE Transactions on Medical Imaging. 2021.
[4] Q. Zou, A. Ahmed, P. Nagpal, S. Kruger, M. Jacob, Dynamic imaging using deep generative STORM (Gen-STORM) model, IEEE Transactions on Medical Imaging.
[5] Rusho, R. Z., Zou, Q., Alam, W., et al. Accelerated Pseudo 3D Dynamic Speech MR Imaging at 3 T Using Unsupervised Deep Variational Manifold Learning. Medical Image Computing and Computer Assisted Intervention—MICCAI 2022. Lecture Notes in Computer Science, vol 13436.
[6] X. Peng, F. Lam, A Generative Subspace Model for High-dimensional MR Imaging, the 31st Annual Meeting of ISMRM, Toronto, Canada, 2023, p 0860.

What is claimed is:

1. A method for phase-contrast magnetic resonance image reconstruction, comprising:
receiving undersampled data from a plurality of phase-contrast magnetic resonance image acquisitions;
initializing a deep generative neural network with network parameters and acquisition-specific input vectors;
iteratively modifying the network parameters and input vectors to minimize a cost function to provide a modified network, wherein the cost function comprises a data consistency term between the undersampled data and an output of the deep generative neural network, an anatomical consistency term that enforces structural similarity across the plurality of acquisitions, and a physics-based term that enforces properties of phase variations; and
generating reconstructed phase-contrast magnetic resonance images using the modified network.

2. The method of claim 1, wherein the anatomical consistency term penalizes differences between magnitude images from different acquisitions.

3. The method of claim 1, wherein the physics-based term enforces at least one of: spatial smoothness or temporal smoothness of phase components; divergence-free properties of vector fields; or harmonic properties of displacement fields.

4. The method of claim 1, wherein the cost function further comprises an input vector constraint term wherein the input vector is fixed as spatiotemporal coordinates of the targeted image function.

5. The method of claim 1, wherein each of the plurality of phase-contrast magnetic resonance image acquisitions represents: a different time point; a different encoding direction; or a different phase of motion.

6. The method of claim 1, wherein the deep generative neural network maps between a low-dimensional latent space and image space.

7. The method of claim 1, wherein the method is applied to at least one of: magnetic resonance elastography; magnetic resonance flow imaging; or magnetic resonance thermometry.

8. The method of claim 1, further comprising extracting quantitative physiological information from the reconstructed phase-contrast magnetic resonance images.

9. The method of claim 8, further comprising providing the quantitative physiological information for clinical diagnosis.

10. The method of claim 1, wherein the physics-based term comprises application-specific properties including: for elastography: properties of mechanical wave propagation; for flow imaging: properties of fluid dynamics; or for thermometry: properties of heat diffusion.

11. The method of claim 1, wherein iteratively modifying comprises alternating between: updating the network parameters while holding the input vectors fixed; and updating the input vectors while holding the network parameters fixed.

12. A non-transitory computer-readable medium storing instructions that, when executed by a computing device, cause the computing device to perform the method of claim 1.

13. The method of claim 1, wherein the deep generative neural network represents an image at a specific acquisition as a function of the parameters and an acquisition-specific input vector.

14. The method of claim 1, wherein iteratively modifying the network parameters and input vectors comprises an unsupervised learning process that estimates the parameters and input vectors directly from the undersampled data.

15. A system for reconstructing phase-contrast magnetic resonance images, comprising: a magnetic resonance imaging device configured to acquire undersampled data; and a computing device configured to perform the method of claim 1.

16. A method for accelerated phase-contrast magnetic resonance imaging, comprising:
receiving undersampled data from a magnetic resonance imaging scanner;
initializing a deep generative neural network with parameters and a set of acquisition-specific input vectors;
iteratively:
generating trial images by processing the set of input vectors through the deep generative neural network;
computing a cost function value based on data consistency, anatomical similarity across acquisitions, and physics-based constraints;
updating the parameters and input vectors to minimize the cost function value; and
outputting reconstructed phase-contrast images generated by the updated deep generative neural network.

17. The method of claim 16, wherein the deep generative neural network shares common network parameters across all acquisitions while using a different input vector for each acquisition; and wherein in a special case, the input vector is fixed as spatiotemporal coordinates of a targeted image function.

18. The method of claim 16, wherein the cost function penalizes differences between magnitude images of different phase-contrast acquisitions while allowing phase variations between the different phase-contrast acquisitions.

19. The method of claim 16, wherein the parameters of the deep generative neural network are determined without using pre-acquired training datasets.
